(19) United States
(12) Patent Application Publication
(10) Pub. No.: US 2019/0090775 A1
(43) Pub. Date: Mar. 28, 2019

(54) Physiologically Modulated Visual Entrainment Device

(71) Applicant: Advanced Health & Performance Institute, LLC, Orlando, FL (US)

(72) Inventors: Adrian E. Quevedo, Longwood, FL (US); Richard G. Soutar, Woodstock, GA (US)

(21) Appl. No.: 15/982,730

(22) Filed: May 17, 2018

Related U.S. Application Data
(63) Continuation of application No. 12/613,486, filed on Nov. 5, 2009, now abandoned.
(60) Provisional application No. 61/111,810, filed on Nov. 6, 2008.

(51) Int. Cl. A61B 5/0482; A61B 5/00; A61N 5/06; A61B 5/04; A61B 5/021

(52) U.S. Cl. CPC A61B 5/0482; A61B 5/411; A61N 5/0618; A61B 5/6814; A61B 5/441; A61N 2005/0652; A61B 5/04012; A61B 5/021; A61N 2005/0648

(57) ABSTRACT

A device comprised of one component that emits pulsing light patterns across the entire human visual field and a second component that monitors physiological activity and regulates the first component. The device is composed of glasses with dense light array emitting sources across the entire visual field of both eyes. This array may emit any of the possible combinations and permutations from the domains of (but not limited to), wave frequency, intensity, color, coherence, phase and type including sinusoidal, square, or saw tooth waves. By driving these possible combinations, different areas of the brain can be stimulated. Since various areas of the brain control CNS (Central Nervous System) functions, the secondary CNS monitoring component can be coupled with these various brain regions to enhance response to training. The data that the device detectors collect will provide recommendations for natural and pharmaceutical agents to enhance the function of the device.

### CROSS REFERENCE TO RELATED APPLICATION

This application is a continuation of U.S. application Ser. No. 12/613,486 filed Nov. 5, 2009, now abandoned, which claimed priority to U.S. provisional patent application 61/111,810 filed Nov. 6, 2008.

### BACKGROUND OF THE INVENTION

This invention uses photic stimulation to stimulate the Central Nervous System (CNS). The focus of this invention is to cause shifts in brainwave dynamics with more specificity such that certain regions of the brain can be more affected rather than just globally or by hemisphere like previous art attempts to accomplish. It is used in conjunction with a set of glasses, including a wrist band device, finger band device or other peripherals to monitor CNS arousal levels and modulate the patterns of the light emitting sources to stimulate activity to enhance brain function under different conditions from low arousal relaxed states to higher arousal focused states. This other added feature provides a bottom up effect in driving the CNS as well as a top down effect. The goal is to exercise different areas of the brain to achieve maximum efficiency of function. This invention also uses the data collected from the detectors on the device to derive a protocol regimen of natural or pharmaceutical agents to be taken by the user once the arousal levels have been determined by the device. Science has made significant progress in the understanding of how the brain and central nervous system functions. The physiology of the brain and how it is related to various aspects of cognitive function have also started to become clear. This understanding of cognitive functions includes focus, concentration, memory, as well as, central nervous system disorders such as Alzheimer's Dementia to Parkinson's Disease. This advancement in our scientific understanding also extends to the underlying factors that contribute to changes in physiology that are causal to the nature of the disorder. This is significant in that it allows for the development in oral therapies, both natural and pharmaceutical agents that can target needed areas in the brain and neural tissues to drive the physiology to a higher order of function.

Pharmaceutical agents are often designed to target metabolic factors that are driving negative aspects of the physiology affecting cognitive functions. For example, ARICEPT® (donepezil) functions by inhibiting acetylcholinesterase activity, which when elevate degrades acetylcholine levels, a transmitter in the brain that is necessary for functional memory. The inhibitory nature of most synthetic pharmaceutical agents can now be safely strategized with natural agents in a consorted effort to achieve the desired responses to cognitive functions. Natural agents operate alternatively as agonists to their relative metabolic functions. This has been described by a professional movement of physicians who practice orthomolecular medicine. According to the movement's most significant professional association, Ortho Molecular Medicine Online: “Orthomolecular medicine, as conceptualized by double-Nobel laureate Linus Pauling, aims to restore the optimum environment of the body by correcting imbalances or deficiencies based on individual biochemistry, using substances natural to the body such as vitamins, minerals, amino acids, trace elements and fatty acids. The term “orthomolecular” was first used by Linus Pauling in a paper he wrote in the journal Science in 1968. The key idea in orthomolecular medicine is that genetic factors affect not only the physical characteristics of individuals, but also to their biochemical milieu. Biochemical pathways of the body have significant genetic variability and diseases such as atherosclerosis, cancer, schizophrenia or depression are associated with specific biochemical abnormalities which are causal or contributing factors of the illness.”

Further, there are a number of plausible mechanisms by which the concentration of a micronutrient can affect the functioning of the brain. Described by Dr. Linus Pauling (Science, 1968) as follows: “There are a number of plausible mechanisms by which the concentration of a vitamin may affect the functioning of the brain. One mechanism, effective COT vitamins that serve as coenzymes, is that of shifting the equilibrium for the reaction of apoenzyme and coenzyme to give the active enzyme. An example is the effectiveness of cyanocobalamin (vitamin B12) given in amounts 1,000 times greater than normal to control the disease methylmalonic aciduria (Orsenberg et al 1968; Lindblad, et al 1968; Walker et al, 1969; Rosenberg, et al 1969). About half of the patients with this disease are successfully treated with megadoses of vitamin B12. In these patients a genetic mutation has occurred and an altered apoenzyme that has a greatly reduced affinity for the coenzyme has been produced. Increase in concentration of the coenzyme can counteract the effect of the decrease in the value of the combining constant and lead to the formation of enough of the active enzyme to catalyze effectively the reaction of conversion of methylmalonic acid to succinic acid. In the human population there may be several alleles of the gene controlling the manufacture of each apoenzyme; in consequence the concentration of coenzyme needed to produce the amount of active enzyme required for optimum health may well be somewhat different for different individuals—In particular, many individuals may require a considerably higher concentration of one Or more coenzymes than other people do for optimum health, especially for optimum mental health. It is difficult to obtain experimental evidence for gene mutations that lead to only small changes in the properties of enzymes. The fact that genes that lead to large and more easily detectable changes in the properties of enzymes occur, as in individuals with methylmalonic aciduria, for example, suggests that mutations that lead to small changes also occur. Significant differences in enzyme activity in different individuals have been reported by many investigators, especially by Roger Williams Ph.D. (Williams, R. J.: Biochemical Individuality. New York, John Wiley & Sons, 1957), who has made many studies of biochemical individuality. It is likely that thorough studies of enzymes would show them to be similar to the human hemoglobins. A few of the abnormal human hemoglobins, most of which involve only the substitution of one amino-acid residue for another in either the alpha chain or the beta chain of the molecule, differ greatly in properties from normal adult hemoglobin, leading to serious manifestations of disease.”

These therapies include a large and growing pharmacopeia of natural agents that drive metabolisms towards optimal balance, leading to improved function. This has also been described using Le Chatelier's principle (http://en.wikipedia.org/wiki/Le_Chatelier %27s_principle), which states that a sluggish metabolic system (causing dysfunction) can be flooded with micronutrient substrates to push those metabolisms towards an equilibrium or homeostasis. In other words, improving function by driving a sluggish metabolism towards equilibrium through mass action with oral micronutrient therapies, thus equalizing the system.

The following represents proposed “Mechanisms of Action” supported throughout the scientific literature with regards to cognitive, brain, and neural tissue function. This further serves as the basis for developing targeted oral therapeutics by correlating these mechanisms with known physiology and traditional DSM-IV classifications.

A broader view of nutritional deficiency is a relative state of insufficiency due to lifestyle and environmental factors challenging availability and usage by the body. This is evidence by clinical observations of individuals who represent with clinical signs of deficiency even though blood levels of these nutrients represent as adequate for proper biological function. This micro nutrient insufficiency has been observed and related to risks for Dementia. For example, studies have found that a high intake of fish oil (omega 3 fatty acids EPA & DHA) was associated with a significantly lower risk of (Kalmijn V et al 1997). These findings have been supported in several other studies (Solfrizzi V et al 2005; Solfrizzi V et al 2003; Solfrizzi V et al 1999; Panza F et al 2004; Capurso A et al 2000).

Oxidative stress is fundamentally involved in the pathophysiology of every disease known to man. The brain, being a high energy organ, is even more prone to oxidative damage. The body receives protection from this damaging free radical stress by the quenching power of both primary antioxidants (i.e. SOD, Glutathione Peroxidase) and secondary (nutritional) antioxidants. Animal Studies have shown evidence that diets high in antioxidants delay age-associated memory loss (Joseph J A et al 1998; Perrig W J et al 1997).

Scientists have been championing a theory that links inflammation to cognitive decline, Alzheimer's Dementia, Parkinson's Disease, as well as, other chronic central nervous system disorders. Several studies have examined the association between chronic inflammation and mild cognitive impairment. For example one study 2632 men found that those who had high inflammation levels were more likely to experience cognitive impairment (Yaffe K et al 1998).

Methylation deficits (insufficiency of vitamin B6, Folic Acid, Vitamin B12, & Trimethylglycine) Insufficiency of methylating nutrients can lead to elevated blood levels homocystiene, a toxic byproduct of methionine metabolism. Leading cardiovascular researchers believe that homocysteine damages the arteries and is an independent risk factor for atherosclerosis. This plague building process reduces blood flow to the brain and may also contribute to the formation of beta-amyloid build up in the brain. One study found that elevated levels of homocysteine is associated with reduced processing capacity and speed of information processing (Aleman A et al 2005).

A new theory proposes a strong relationship between stress and cognitive impairment. Studies have found that older men with elevated levels of stress hormones (epinephrine) are more likely to suffer from mild cognitive impairment then their piers with normal levels (Karlamangla A S et al 2005). It's also been shown that everyday stress combined with major stressful events can have a cumulative effect over a lifetime that speeds up cognitive decline (VonDras D D et al 2005).

Elevated fibrinogen levels contribute to the tendency to form dangerous clots that reduce blood flow to the brain causing mild cognitive impairment (Carmeliet. 1995). This state of excessively think and dotty blood negatively affects its dynamics causing endothelial dysfunction, damages the vasculature, and reduces circulation of oxygen and nutrients to the brain.

Mild to moderate cognitive impairment has been observed in chronic allergy (Classic IgG & Delayed onset IgG) suffers. Regulation of hyper immune reactions may protect the brain from inflammatory insults on the glial cells and neurons.

The brain is one of the body's most energy intensive organs. Persistent energy deficits caused by mitochondrial dysfunction leads to metabolic disorders that can cause mild to moderate cognitive decline. Many researchers now consider this to be one of the critical early onset factors of Alzheimer's and Parkinson's Disease.

Nitric oxide (NO) deficits can contribute to mild to moderate cognitive decline. NO dysfunction reduces circulation and immune function in the brain.

Mild cognitive impairment has been observed in neurons that have challenged cellular membrane integrity. One example of this effect is on the function and activity of neurotransmitters as they fire across the synaptic cleft. As cellular membranes lose their integrity, they succumb to more oxidative stress and other insults that cause them to further uncouple and become dysfunctional. This leads to poor neuronal activity, especially affecting the function of receptors for neurotransmitters, which reduces cognitive functions. A couple of cellular conditions include; Cellular Detoxification (i.e. lypofuscan, Tau protein, and beta-amyloid) and Cellular Rejuvenation (i.e. growth factors). Finally, Central Nervous System Modulation (Neurotransmitter function—Inhibitory Vs Excitotory) and Endocrine Modulation (Sympathetic Vs Parasympathetic, circadian rhythms) are contributors to cognitive disfunction.

Several devices such as U.S. Pat. No. 4,315,502 teach of a device for generating luminous stimuli for the eye, comprising a housing that can be fixed in front of the user's face. U.S. Pat. No. 5,709,645 presents a photic stimulator capable of stimulating the left and right visual fields of each eye independently of each other. It provides an apparatus for stimulating the central nervous system and the brain waves of a human subject having left and right eyes and left and right visual fields within each eye. U.S. Pat. No. 5,242,376 describes a relaxation device that uses a portable mask that is positioned on a subjects head and uses a flashing light source to relax the user through the eyes. U.S. Pat. No. 6,299,632 is an apparatus for stimulating cortical brain activity in the non-dominant hemisphere by using light or sound in close proximity to the patient's eyes. U.S. Pat. No. 5,306,228 uses light and sound to shift the brain into a predetermined frequency set by the device. U.S. Pat. No. 4,858,609 is a bright light mask system for shining high intensity light into subject's eyes to modify circadian rhythms. U.S. Pat. No. 5,146,927 uses a method of determining the functional integrity of the visual system if visually impaired human beings. The method includes measuring melatonin content in blood samples taken before, during, and after application of the stimulus. U.S. Pat. No. 5,954,640 is a method for providing nutritional supplementation for a person using information relating to health and diet. This method also considers a budget for the nutritional supplementation. U.S. Pat. No. 6,081,743 is a method for treating individuals using EEG feedback of the highest evoked potential in the brain to determine the appropriate brainwave frequency to train to the highest evoked response of the individual. And finally, U.S. Pat. No. 4,852,570 is a medical process and apparatus for repeatedly obtaining short term changes in the physiological functioning of an individual as an aid in diagnosing illness and malfunction. It also uses apparatus that can include multiphasic diagnostic equipments that perform the numerous tests at frequent intervals.

### BRIEF SUMMARY OF THE INVENTION

The invention recognizes the importance of all possible combinations and permutations of the entire visual field, as discussed in the general neurology texts, and is devised to work on different regions of the brain associated with different arousal levels while monitoring arousal level to insure that those regions are functioning optimally for those specific arousal levels. FIG. 1 shows one of the possible combinations and permutations of light emitting source placement. The feedback provided by the monitoring device in conjunction with light stimulation that varies in (but not limited to), wave frequency, wave intensity, wave color, wave coherence, wave phase and wave type including sinusoidal wave, square wave, or saw tooth wave, allows stimulation of specific areas of the brain to exercise networks and improve efficiency and function.

The monitoring devices review electromagnetic activities that are proxy measures of various physiological processes. Research shows these different physiological activities are correlated with different brainwave patterns and that normative relationships exist between these physiological patterns and brainwave activity. Together, the feedback loops between lower and higher CNS networks can be mutually adjusted to normalize the CNS through carefully controlled light stimulation utilizing various sections of the eyefields.

One purpose of the present invention is to provide physiologically modulated light stimulation of various combinations of regions of the eyefields to stimulate various specific brain regions with considerable specificity. The light stimulation provides further specificity with regard to brainwave frequency control by also modulating combinations of (but not limited to), wave frequency, wave intensity, wave color, wave coherence, wave phase and wave type including sinusoidal wave, square wave, or saw tooth wave.

Physiological monitoring begins when the system is activated. The wrist device, finger device or glasses picks up electromagnetic signals and skin conductance information from electrodes attached to the wrist band, finger device or glasses or other peripheral detector. These electromagnetic signals can be analyzed as proxy measures of physiological activity depending on their frequency range. The signals are amplified by a differential preamplifier to filter background noise through phase cancellation and then digitally filtered and further processed by a series of amplifiers. The resulting signals are then sent to a microprocessor that filters the signals into component bands that are proxy measures of various physiological processes including heart rate, heart pulse, muscle tension, skin temperature, skin conductance, and EEG. These signals are analyzed to determine if they are in the normal operational range, the resulting data is transmitted to the glasses. These ranges are shown below.

Electro-Magnetic Outputs

| | Low(Normal) | | Moderate | | Hi | |
|---|---|---|---|---|---|---|
| EMG | 1-5 | uv | 5-15 | uv | 15-25 | uv |
| SCR | 3-8 | umho | 8-16 | umho | 16-24 | umho |
| TMP | 90-95 | F. | 85-90 | F. | 75-85 | F. |
| (Temperature) | | | | | | |
| ECG (Pulse) | 65-80 | bpm | 80-100 | bpm | 100-130 | bpm |
| Blood Pressure | 120/80 | mm hg | 140/90 | mm hg | 160/100 | mm hg |
| EEG | | | | | | |
| Delta | 10 | uv | 15 | uv | 20 | uv |
| Theta | 8 | uv | 13 | uv | 18 | uv |
| Alpha | 6 | uv | 10 | uv | 16 | uv |
| Beta | 4 | uv | 8 | uv | 12 | uv |
| HRV | | | | | | |
| Negative | 0-.5 | Hz | | | | |
| Sympathetic | | | | | | |
| Positive | .5-.15 | Hz | | | | |
| Sympathetic | | | | | | |
| Parasym- | .15-.25 | Hz | | | | |
| pathetic | | | | | | |

uv = microvolt
Umho = micromho
F. = Fahrenheit
bpm = beats/minute
mm Hg = millimeters of Mercury
Hz = Hertz

A photic stimulation session begins when the glasses respond by initiating pulse patterns in various configurations across the visual field. These pulse patterns vary in color, intensity, frequency, etc. depending on the incoming data regarding physiological activity. This information is transferred to the optic nerve and then to the lateral geniculate nucleus. From there it transfers to the occipital cortex and thalamacortical loops impacting various regions of the brain. The light patterns in the glasses adjust to impact brain regions that will normalize physiological activity. As physiological activity changes, the watch device, wrist device or glasses read this activity and informs the glasses to alter their pattern accordingly to further normalize physiological activity. After a specified pre-programmed time period the program stops and the glasses stop operating.

### BRIEF DESCRIPTION OF THE SEVERAL VIEWS OF THE DRAWINGS

FIG. 1-A shows a frontal view of the frame (101) of the Glasses (100) with a Nasion Detector (103) in dotted lines indicating an internal feature. FIG. 1 also shows the eyefield and inclusive Light Emitting Source (114) placement within inner (119), middle (118) and outer quadrants (117) of the eye field. FIG. 1-A also shows a dotted square indicating the exploded view area of FIG. 1-B. FIG. 1-B shows on the left lens, in one quadrant of the eyefield, one possible combination and permutation of how the Light Emitting Sources (114) can be placed in 1 of 4 quadrants for each eye. FIG. 2 shows a right sided view of the glasses with internal features including the Battery or Power Source (111), Wireless Network Circuits (112), Network Circuits (113) and the Right Mastoid Detector (104) at the tip of the arm. FIG. 2 also shows the USB/Charging Port (110) at the bottom and is also shown in FIG. 4-A from a bottom view. FIG. 3 shows the left side of the glasses with internal components including a Memory Card (108), Left Mastoid Detector (105) and a Soft key (109) that is also shown in FIG. 4-A. FIG. 4-A also illustrates a dotted square indicating the exploded view in FIG. 4-B to assist in visualization of the dense light array including one Light Emitting Source (114).

FIG. 5 shows the top view of the Wrist Band Device (200). Internal components including the Network Circuits (209), Battery or Power Source (210) and Wireless Network Circuits (208) are represented by dotted lines. 4 Soft Keys (202), a Display (206), and both Adjustable Strap (204) and Receiving Strap (205) that are lined with Strap Detectors (212) are also shown in this figure. FIG. 6 shows a left sided view that exposes the Memory Card Slot (207) and shows other features already shown in FIG. 5. FIG. 7 exposes the USB/Charging Port (203) and other features already shown in FIG. 5.

FIG. 8 shows a top view of the Finger Device (300). The Wireless Network Circuits (305), Network Circuits (306), USB/Charging Port (303) and a Battery or Power Source (307) are all represented by dotted lines to indicate internal components. 4 Soft Keys (302) and a Display (304) are visible in this view. 2 Finger Bands (301) made of a stretchable material are also represented in this view. FIG. 9 is a side view of already mentioned features in FIG. 8. FIGS. 10 and 11 are front and back views of the Finger Device. Electrically Conductive Material (308) is shown lining the inside of the Finger Bands (301) in both FIGS. 10 and 11. In FIG. 10 the USB/Charging port is exposed. In FIG. 11, the Memory Card Slot (309) can be seen.

FIG. 12 shows a computing device (400). The device may use Wireless Network Circuits (401) capable of pairing with the Glasses, Wrist, and Finger devices to communicate, upload, download or update data to and from the devices. The device may also communicate with the peripherals via Memory Card Slots (402) or USB ports (403)

FIG. 13 is a flow chart for the Oral Therapeutics Determinant Process or OTDP.

100) Glasses 101) Photic Stim Glasses Frame 102) Photic Stim Lens 103) Nasion Detector 104) Right Mastiod Detector 105) Left Mastiod Detector 106) Right Arm 107) Left Arm 108) Memory Card 109) Soft Key 110) USB/Charging Port Material 111) Battery or Power Source 112) Wireless Network Circuits 113) Network Circuits 114) Light Emitting Source 115) Lens Detector 116) Arm Detector Detector 117) Outer Quadrant 118) Middle Quadrant 119) Inner Quadrant 200) Wrist Device 201) Wrist Device Housing 202) Soft Key 203) USB/Charging Port 204) Adjustable Strap 205) Receiving Strap 206) Display 207) Memory Card Slot 208) Wireless Network Circuits 209) Network Circuits 210) Battery or Power Source 211) Sound Component 212) Strap Detector 213) Wrist Device Syrface Detector 300) Finger Device 301) Finger Band 302) Soft Key 303) USB/Charging Port 304) Display 305) Wireless Network Circuits 306) Network Circuits 307) Battery or Power Source 308) Electrically Conductive Material 309) Memory Card Slot 400) Computing Device 401) Wireless Network Circuits 402) Memory Card Slot 403 USB Port

### DETAILED DESCRIPTION OF THE INVENTION

One novel aspect of the invention is its method of stimulating combinations of the eyefields of each eye using a variety of (but not limited to), wave frequency, wave intensity, wave color, wave coherence, wave phase and wave type including sinusoidal wave, square wave, or saw tooth wave.

The stimulation of various combinations and permutations of the entire eyefield is used in order to specifically target different regions and networks of the brain for enhancing brain function efficiency by causing shifts in brainwave dynamics with high specificity such that certain regions of the brain can be more affected rather than just globally or by hemisphere. These shifts have been documented to cause changes in structure and function of neural tissue and associated networks.

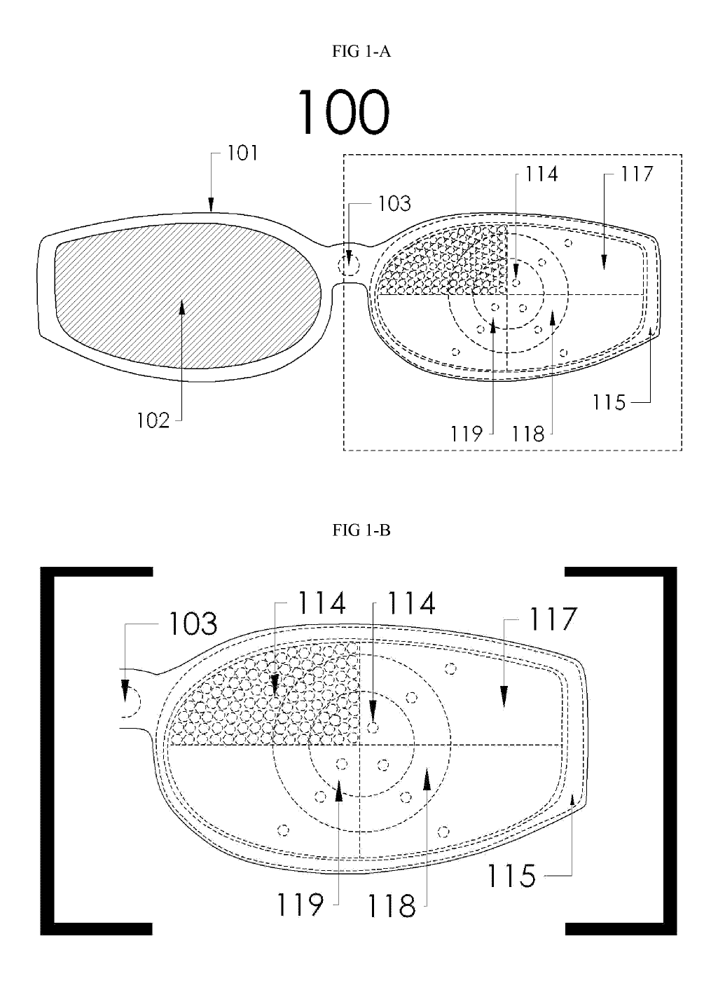

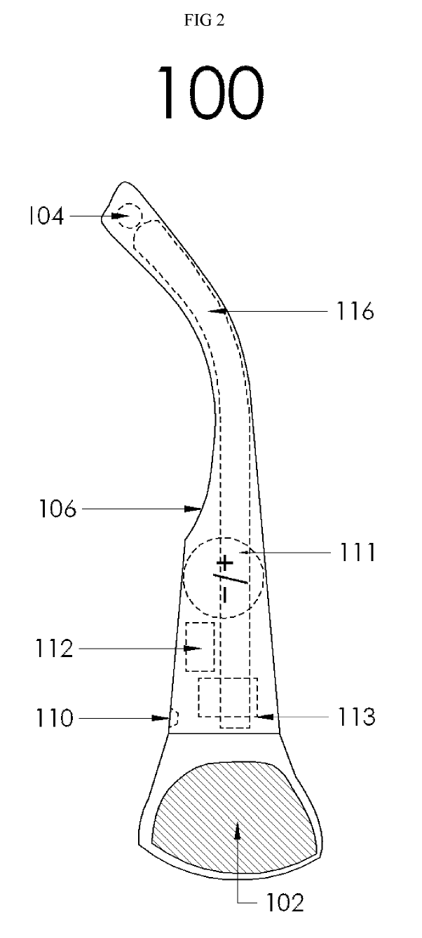

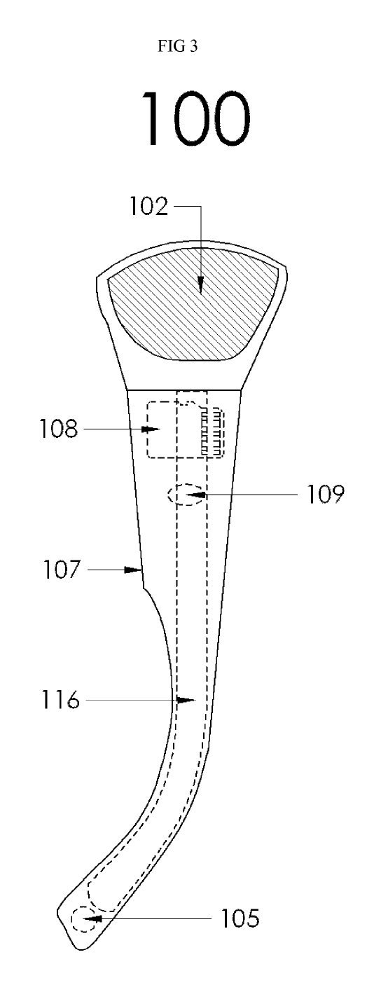

An embodiment of this function includes these components. 1) A set of Glasses (100) with embedded Light Emitting Sources (114) in both lenses covering the entire visual field which can range from a portion, to the entire field. FIGS. 1-A and 1-B (114) 2) Electronics mounted in the frames for modulating the Light Emitting Sources (114) with respect to (but not limited to), wave frequency, wave intensity, wave color, wave coherence, wave phase and wave type including sinusoidal wave, square wave, or saw tooth wave. FIG. 2 (113) 3) Wireless Network Circuits for radio communication with the Wrist Device (200), Finger Device (300), Glasses (100), Computing Device (400) and other peripherals associated to the device. FIG. 2 (112) 4) Battery or Power Source mounted in the frames. FIG. 2 (111) 5) Electrodes mounted in the frames for reading physiological signals. FIGS. 1,2, and 3 (103,104, 105, 115, and 116) 6) A USB/Charging Port (110) for communication with peripherals or computing devices and for charging the internal Battery or Power Source. FIGS. 2 and 4-A (110)

The invention will become active with a 2 step process. First the user will press the Soft Key (109) on the Glasses (100) (FIGS. 3 and 4-A) to switch them to the ON setting and initiate the available mode. The available mode is the setting on the Glasses (100) which allows other peripheral devices to recognize it is ready to pair. Once the glasses are operational, the user will press a specific Soft Key (202) and (302) on the peripheral device (200) and (300) to initiate the search mode. Search mode is a setting on that initiates communication with another device in available or search mode. Once the devices are paired, the user will put the Glasses (100) on. A paired device has accepted communication with another device until the end of a session or a device physically falls out of the range of the wireless communication or a specific soft key is pressed to disconnect or terminate paring. The wireless system will be a means by which the collected information from the peripheral devices will be transmitted to the Glasses (100) to modulate the behavior of the Glasses (100).

The invention will gather physiological data such as Electroencephalograph (EEG), All forms of EDR″ (Electro-Dermal Responses) including Skin Conductance (SCR), Heart Rate Variability (HRV), ECG (Electrocardiogram) Blood Pressure, etc., through the detectors (103,104, 105, 115, and 116) in the Glasses (100) or other peripherals and transmit it to the peripheral device (200 and 300) or it can process the data itself. This data will contribute to the physiological data such as EEG, SCR, HRV gathered at the location of the peripheral devices (103, 104,105,115, 116, 212, 213, and 308). The receiver (112) in the Glasses (100) will receive information from the peripheral devices (200 and 300) or from its own detectors (103, 104,105, 115, and 116) and will initiate an excitation sequence generated by Network Circuits (113) embedded in the Photic Stim Glasses Frame (101), Right Arm (106) and Left Arm (107) that control the Light Emitting Sources (114) covering the entire visual eyefield. Various light patterns will be generated involving combinations of locations of Light Emitting Sources (114) in the visual fields. The light sources provide light patterns that emit various combinations and permutations of frequency, phase, coherence, wave form, wave color and intensity of light. The iterative feedback is applied where the combinations of light sources and patterns serve as an input in driving the photic stimulation session design and the physiological monitoring, and in turn, the physiological monitoring serves as an input in driving the combinations of light sources, patterns and in determining the next iteration of the design of the photic stimulation session.

Another aspect of the invention is a device for stimulating the eyes driven by detectors in the device (103,104,105,115, 116) or secondary devices (212, 213, 308) that monitor physiological activity and modulates the visual stimulation based on the physiological activity.

The device for stimulating the eyes (100) will consist of light emitting sources (114) placed on the inside of each lens of the device covering the entire eye field. The device is placed on the face in a similar fashion to sunglasses. The source of the light will be determined by the particular application which determines how each eye will be stimulated using (but not limited to) wave frequency, wave intensity, wave color, wave coherence, wave phase and wave type including sinusoidal wave, square wave, or saw tooth wave patterns.

An embodiment of this function includes these components 1) Network circuits to modulate the wave frequency, wave intensity, wave color wave coherence wave phase and wave type including sinusoidal wave, square wave, or saw tooth way. FIG. 2 (113) 2) Light sources placed across the entire visual field. FIGS. 1-A and 1-B (114) 3) An On/Off soft key to turn the glasses on for pairing with the wrist device or other peripherals. FIGS. 3 and 4-A (109) 4) A Memory Storage Device for collecting data for uploading at a later time. FIG. 3 (108) 5) Detectors placed on the surfaces of the glasses to collect physiological activity such as (but not limited to) EEG, ECG, HRV, SCR etc. FIGS. 1-A. 1-B, 2 and 3 (103, 104, 105, 115) 6) A Battery or power source. FIG. 2 (111) 7) A wireless transmitter/receiver capable of connecting or pairing the Glasses (100), Wrist Device (200), Finger Device (300) or other peripherals. The transmitter would use wireless technology such as Bluetooth or other RF methods. FIG. 2 (112) 8) A USB/Charging port for communication with peripherals (200, 300) or Computing Devices (400) and for charging the internal Battery or Power Source (111). FIGS. 2 and 4-A (110) 9) Soft Key for selecting and setting device functions. FIGS. 3 and 4-A (109).

The user will press the Soft Key (109) on a device (100) to start the session. Once the session begins the device will go into search mode. The user will then place the Glasses (100) on the face in a similar manner to other eyewear. Once the device is paired with either the Wrist (200) device, Finger Device (300) or other peripherals, the receiver in the Glasses (100) will receive information from the peripheral devices (200 or 300) or its own detectors (103,104,105,115, 116) and will initiate an excitation sequence generated by electronics (113) embedded in the frames that control the Light Emission Sources (114) covering the entire visual eyefield. Various light patterns will be generated involving combinations of locations of light sources in the visual fields. The light sources provide light patterns that emit various combinations and permutations of frequency, phase, coherence, wave form, wave color and intensity of light. The iterative feedback of the physiological monitoring serves as an input in driving the combinations of light sources, patterns and design of the photic stimulation session. The above mentioned patterns of the lights will adjust according to the inputs from the detectory devices on the Glasses (103,104,105,115, 116) or from the peripherals (200 or 300). The session will continue until the predetermined time expires. Once the lights are no longer on, the glasses will keep the session stored on the Memory Card (108), on the glasses, Wrist Device Memory Card Slot (207) or any other peripheral. The glasses will remain paired until the session is over, the glasses fall out of the range of other peripherals or the user manually presses the Soft Key (109) to turn the device off.

Another aspect of the invention is the method of communication via wireless systems between the monitoring devices and the device that stimulates the eyes.

The wireless system will serve multiple purposes. The first is to provide bi-directional communication between the Glasses (100), Wrist Device (200), Finger Device (300), and other peripheral devices. Eliminating the wires used with current devices allows for greater ranges of motion and reduces wire clutter. Another function of the wireless system is to provide multiple sources for Glasses (100), Wrist Device (200), Finger Device (300) and other peripheral devices to upload information to. This allows computing device compatibility and gives the user multiple options for processing data, updating software and firmware.

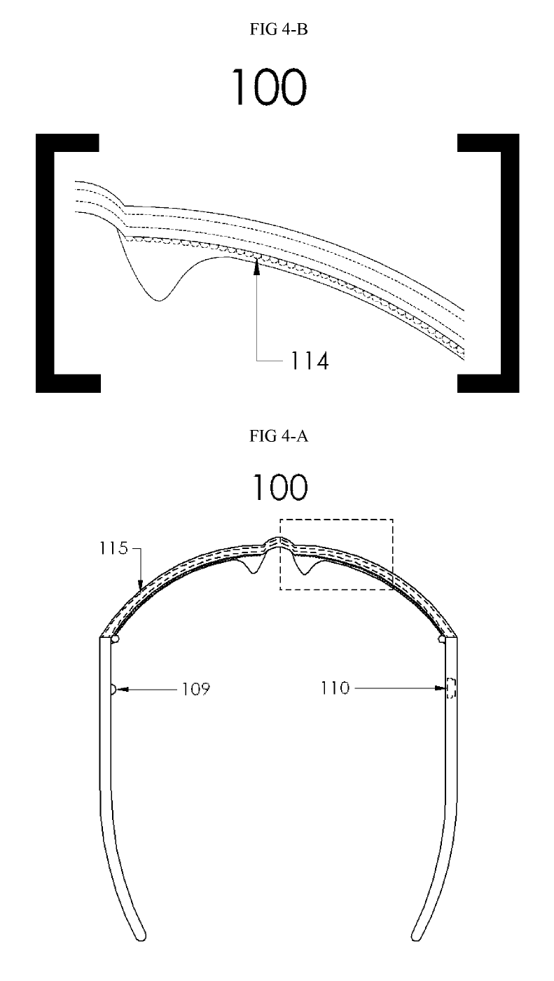

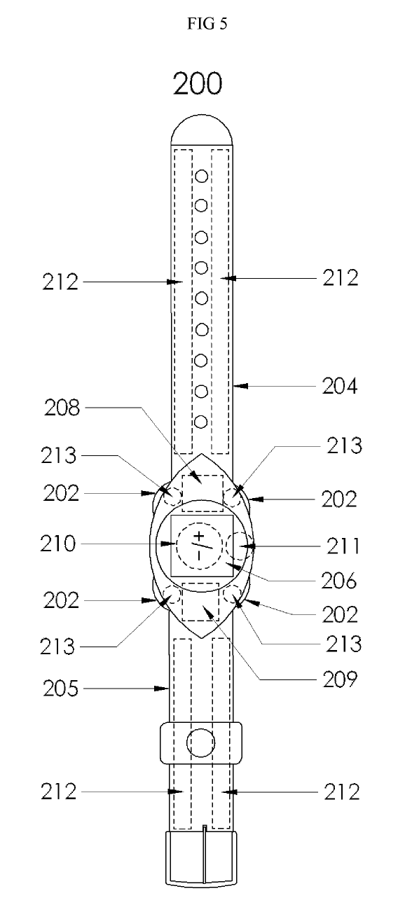

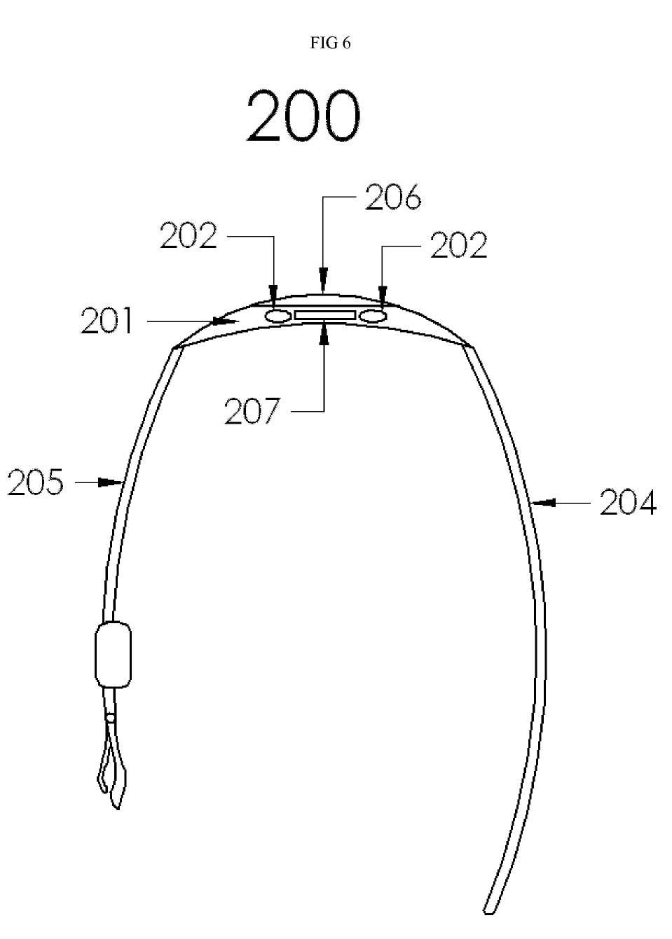

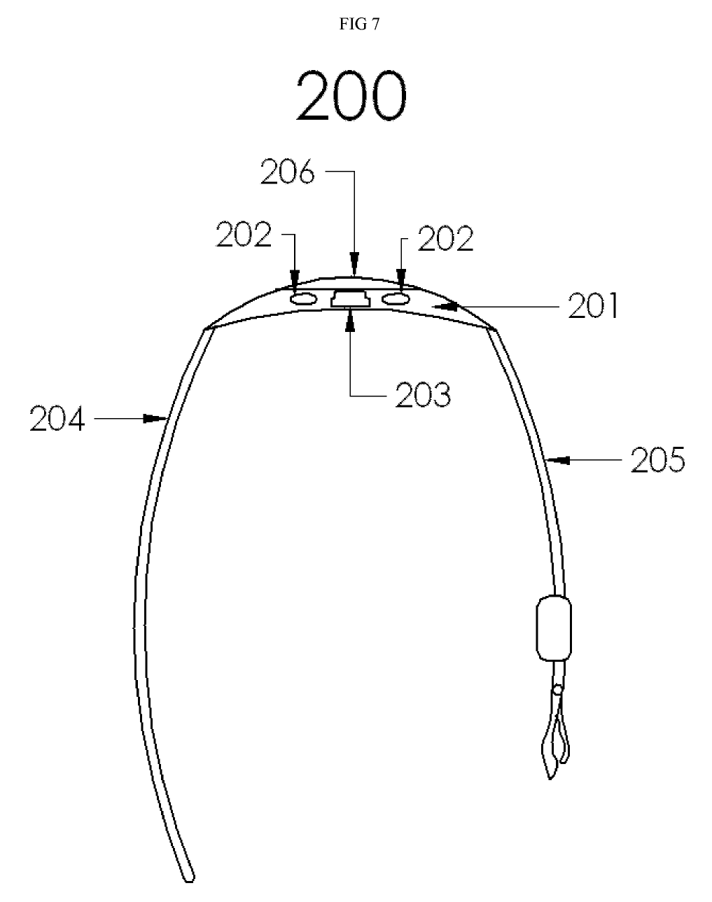

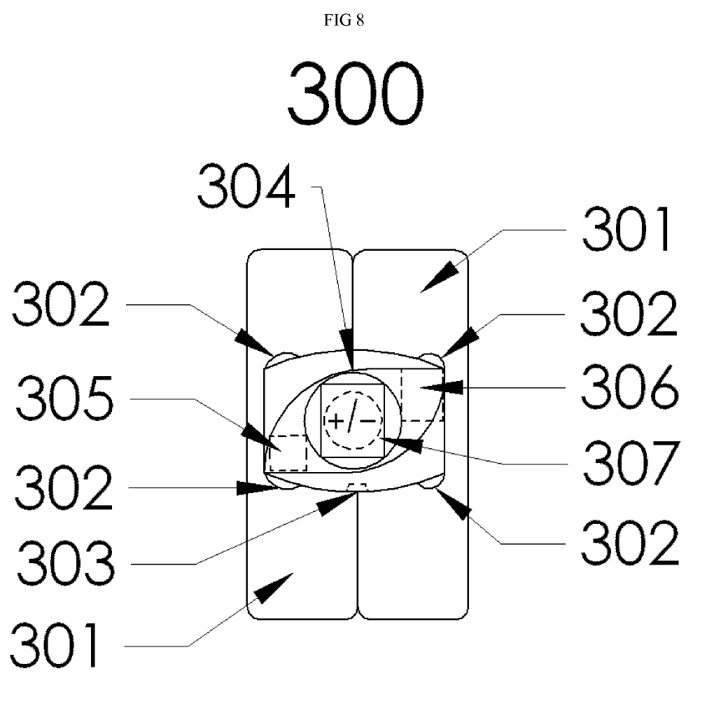

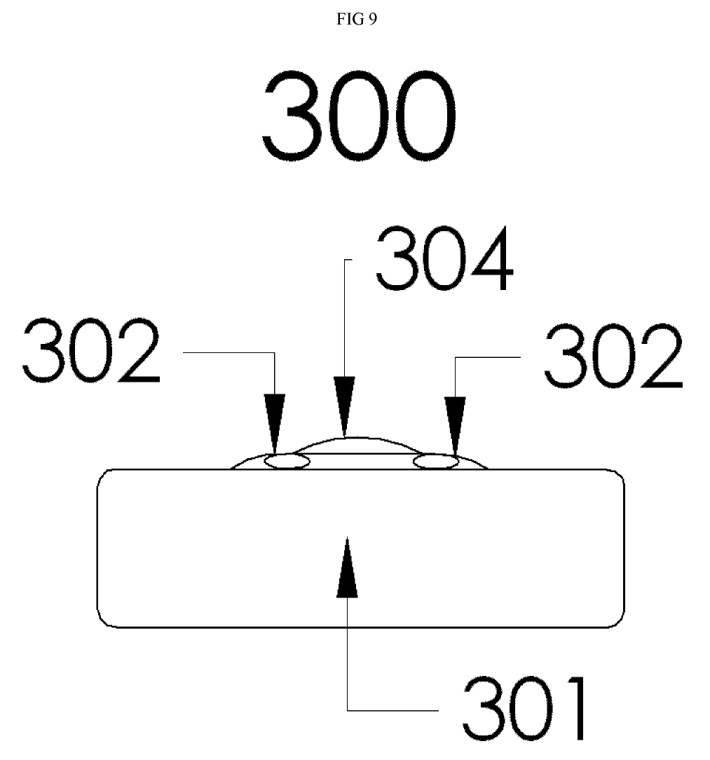

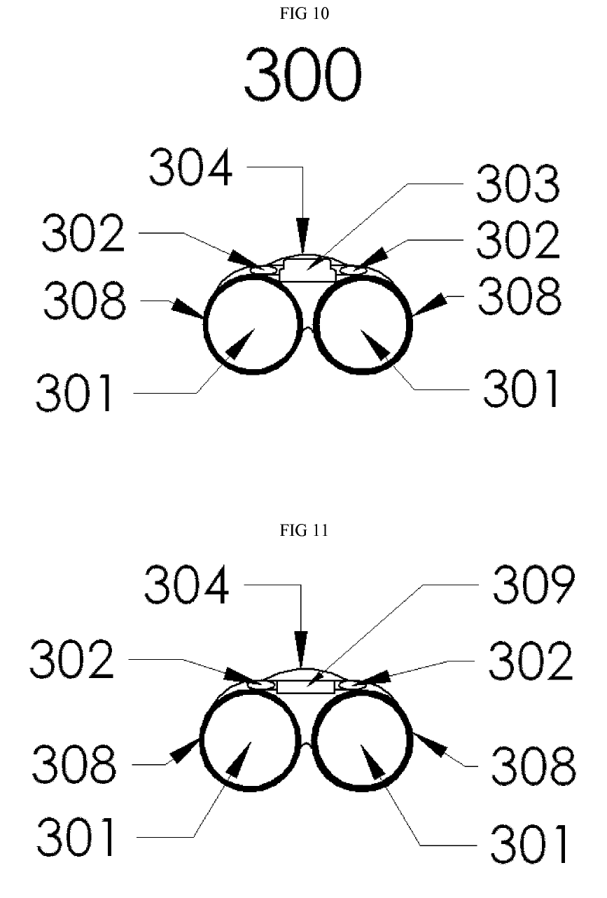

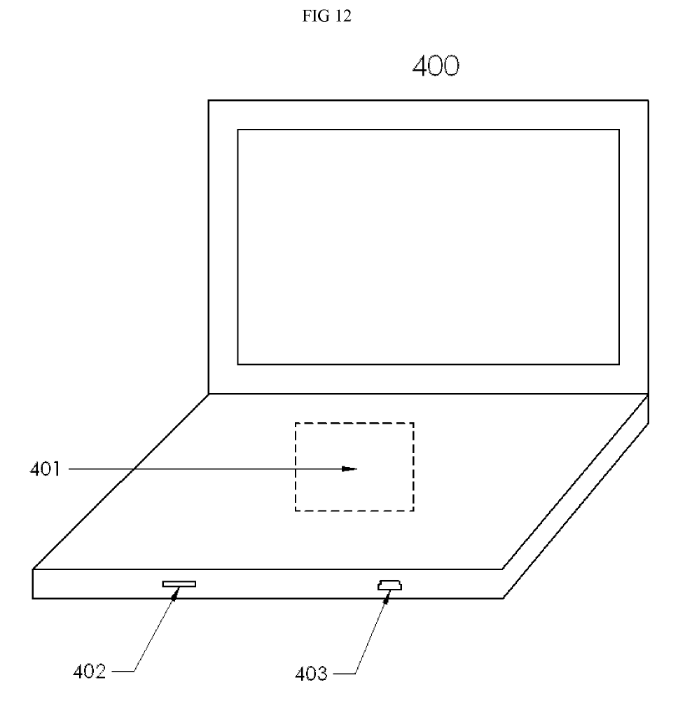

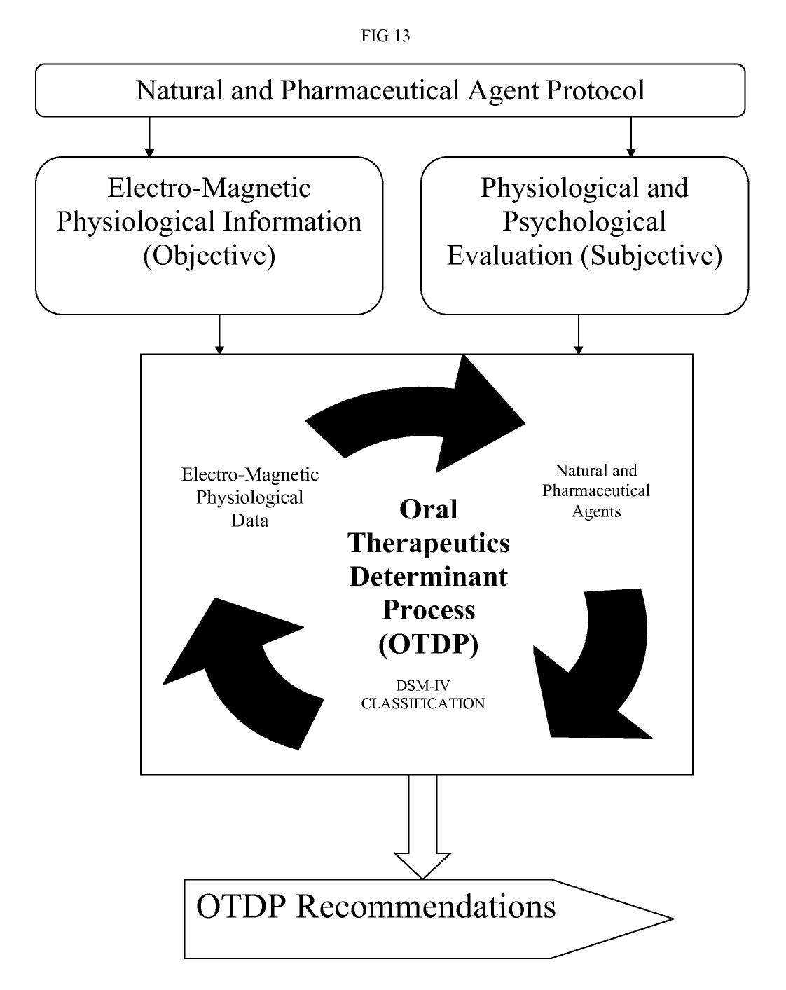

An embodiment of this function includes these components 1) A wireless transmitter/receiver in the peripheral device capable of connecting or pairing with the Glasses (100). The transmitter would use wireless technology such as Bluetooth or other RF methods. FIG. 5 (208) FIG. 8 (305) 2) A wireless transmitter/receiver in the Glasses (100) capable of connecting/pairing with peripheral devices. The transmitter would use wireless technology like Bluetooth or other RF methods. FIG. 2 (112) 3) The wireless system for the Glasses (100) and peripheral devices will also pair in tandem or independently to a Computing Device (400) or other accessories. (i.e. wireless headphones)

The wireless system will become active with a 2 step process. First the user will press the Soft Key on the glasses (109) to switch them to the on setting and initiate the available mode. Once the Glasses (100) are operational the user will press a specific Soft Key (202, 302) on the peripheral device (200, 300) to initiate the search mode. Once the devices are paired, the user will put the Glasses (100) on. During the session the wireless system will iteratively relay the collected data from the detectors (103,104,105, 204,205, and 308) to the peripheral device (200, 300) to then be processed. Once the peripheral device determines the next data set of controls for the Glasses (100) it will send the data packet back to the receiver (112) on the Glasses (100). The network circuits (113) will take the received data and modulate the light frequency, light intensity etc. The wireless system will be the means by which the collected information from the Wrist Device (200), Finger Device (300) or other peripheral will be transmitted to the Glasses (100) to modulate the function of the Glasses (100).

When the session is over the wireless system will remain paired to the Glasses (100) unless the user terminates the connection via pressing a specific soft key, the end of a session occurs or the glasses fall out of the range of the Wrist Device (300) or Finger Device (200). The wireless system will be able to connect the Glasses (100) to other devices (including medical) or physiological detectors in the same manner as described above.

The wireless system will be used to download stored data on the Wrist Device (200) and Glasses (100) or other paired devices to a Computing Device (400) for analysis. The system will also provide a means to upload data to the Glasses (100), Finger Device (200) and Wrist Device (300) or other peripherals for upgrades and maintenance to software or firmware. The USB/Charging Port (110,203 and 303) will also be a means to conduct similar tasks.

Another aspect of the invention is a wrist monitoring device that reads physiological activity from the body and adjusts the visual stimulation accordingly.

The Wrist Device (200) will use a Wireless Network (208) capable of reaching the distance needed to connect wirelessly with the Glasses (100). The Wireless Network (208) will send data to the Glasses (100) determining the (but not limited to) wave frequency, wave intensity, wave color, wave coherence, wave phase and wave type including sinusoidal wave, square wave, or saw tooth wave. The data collected will consist of a single or a combination of physiological sources including but not limited to SCR, ECG, and EEG etc. The Wrist Device (200) uses ports or sockets (203) to accommodate an array of peripherals that can collect and sense physiological outputs from the body. The Glasses (100) will also collect these outputs from its own array of detectors (103,104,105,115, 116) and ones placed on multiple surfaces of the Wrist Device (200) and the Straps (204 and 205) that go around the wrist. The Wrist Device (200) will also store this information on a removable storage device that goes in the Memory Card Slot (207) for the purpose of uploading through the Wireless Network Circuits (208) or physical removal into a Computing Device (400). Along with the transmission of data, the Wrist Device (200) will also receive data from sources like the Glasses (100) and other sources not connected to the watch, for example, EEG, SCR and or ECG detectors on the glasses (103,104 and 105). The Wrist Device (200) will have a Display (206) designed to provide feedback to the user regarding (but not limited to), time, frequency and units of measure related to the functions of the invention.

An embodiment of this function includes these components. 1) Port/plugs to accommodate an array of peripherals and detectors that can collect and sense physiological activity from the body and connect directly through wireless or wired methods to the wrist device. FIG. 7 (203) 2) A Memory Storage Card for storing sessions to be later uploaded via wireless method or physical removal of the card to a computing device. FIG. 6 (207) 3) A Display for real time feedback including but not limited to time, frequency and units of measure. FIGS. 5,6 and 7 (206) 4) A sound component capable of multiple tones to inform or warn the user of important information. FIG. 5 (211) 5) Sensors placed on surfaces to collect physiological activity such as EEG, ECG, HRV, SCR etc. FIGS. 5 (212 and 213) 6) A Battery or Power Source. FIG. 5 (210) 7) Network Circuits for processing iterative data from glasses and finger device or other peripherals. FIG. 5 (209) 8) A wireless transmitter/receiver capable of connecting or pairing with the Glasses (100). The transmitter would use wireless technology such as Bluetooth or other RF methods. FIG. 5 (208) 9) A USB/Charging port for communication with peripherals or computing devices and for charging the internal battery or power source. FIG. 7 (203) 10) Soft Keys for selecting and setting device functions. FIGS. 5,6 and 7 (202)

The user will place the device on the wrist in a similar manner to a wrist watch. The device will already be in a standby mode, preferably with date and time on the screen. Once the user has placed the Glasses (100), Finger Device (200) and/or other peripherals into available mode, the user will press a soft key (202) on the Wrist Device (200) to enter into search mode. The Display (206) will indicate that the search mode has begun. Once the device has paired with the peripheral, a baseline reading consisting of 1 minute of non-modulated physiological data collection will be taken from the peripheral. At the end of the 1 minute baseline, thresholds will be assigned. Thresholds consist of starting point of physiological output used to modulate the light, intensity, etc.

The transmission of the wireless signal will initiate a photic stimulation session. During the session the user can toggle the screen on the Wrist Device with a Soft Key (202) to display information including (but not limited to) light characteristics including frequency, intensity, shape, time remaining, elapsed time, and other chronographic features. The Wrist Device (200) can also display the amount of space on the data storage device, the peripherals paired, and other individual user settings including (but not limited to), name, date of session, duration of session, amount of time spent adjusting thresholds, amount of time above or below thresholds of the baseline

Once the session expires the Wrist Device (200) will enter into an options mode. These options include (but are not limited to) restarting session, choosing another session type, uploading session to a computing device.

Another aspect of the invention is a device used to collect physiological information from the skin surface of the fingers.

A Finger Device (300) will be used to collect the data needed to drive the behavior of the Glasses (100). The device uses bands made of a flexible and adjustable material like nylon or rubber (301) which individually wrap around the fingers. The bands will be lined with an electrically conductive material (308) to measure physiological activity. The wired option will consist of the 2 bands connecting directly to the Watch Device (200) or Glasses (200) in a port or plug (303) made specifically for the Finger Device (300). The wireless option will pair the Finger Device (300) with either the Glasses (100) or the Wrist Device (200) and transmit the data collected using the Wireless Network Circuits (305).

An embodiment of this function will consist of the following components 1) Adjustable finger bands lined with a conductive material. FIGS. 8,9,10 and 11 (301 and 308) 2) A plug that will connect the finger bands to the Watch Device (200) or Glasses (100). FIGS. 8 and 10 (303) 3) A wireless transmitter capable of reaching the distances need to pair with either the Wrist Device (200) or the Glasses (100) independently. FIG. 8 (305) 4) A display. FIGS. 8,9,10, and 11 (304) 5) A Battery or Power Source. FIG. 8 (307) 6) Network Circuits for processing iterative data. FIG. 8 (306) 7) Soft Key for selecting and setting device functions. FIGS. 8,9,10, and 11 (302)

The user will wrap the finger bands (301) around the fingers and adjust accordingly to insure proper fitting. The user will then plug the Finger Device (300) directly to the Wrist Device (200) through the USB/Charging Port (303). If the user chooses to use the wireless function, they can turn on and pair the Finger Device (300) with the Wrist Device (200) or the Glasses (100) by pressing a soft key (302) on the device. The Finger Device (300) will remain paired with the Glasses (100) or Wrist Device (200) until the session is over, the devise is no longer in range or the user presses the soft key (302) to turn the device off.

Another aspect of the invention is a process to determine a recommended protocol of natural and/or pharmaceutical agents to enhance the effectiveness of the device, called the Oral Therapeutics Determinant Process (OTDP). This is accomplished by use of the data collected from all of the detectors including the (but not limited to) glasses, wrist device and finger band device to give recommendations for natural and/or pharmaceutical agents and then have the user concomitantly ingest these oral therapies to further enhance the effectiveness of the device.

Oral therapies include (but not limited to) vitamins, minerals, herbs, homeopathics, nutriceuticals, and/or pharmaceuticals in stand alone or in combination. These oral therapies support metabolisms in the brain that enhance the effectiveness of the device that stimulates the eyes. The purpose is for the devices that measure physiological outputs from the body to measure and correlate the outputs to known deficiencies or functional benefits. Oral therapeutic agents including (but not limited to) vitamins, minerals, herbs, homeopathics, nutricuticals, and/or pharmaceuticals that have known clinical benefits, may be used in combination with the device to enhance its effectiveness. This is accomplished by collecting electro-magnetic physiological information from the user (objective) along with physiological and psychological evaluation (subjective) that is utilized in the OTDP to provide the user a protocol recommendation reported on a daily intake schedule. These oral therapeutic agents work by affecting various metabolisms in neural tissues. The effectiveness of the oral therapeutic agents is enhanced by the device, which uses inputs to make adjustments to the devices outputs brining attention to areas of the brain that need balance. This is accomplished by influencing the brain to bring more oxygen, nutrients, and other regulating factors through increased blood supply to needed areas, therefore enriching the blood with targeted oral therapeutic agents enhances this activity further, increasing the effectiveness of the device.

An embodiment of this function includes these components 1) Collecting electro-magnetic physiological information (objective) from the device. 2) Collect physiological and psychological evaluation information from user (subjective) 3) Inputing both objective and subjective data into the Oral Therapeutics Determinant Process (ODTP), which correlates the data by referencing traditional DSM-IV Classifications with the therapeutic indications for oral natural and pharmaceutical agents. 4) Taking OTDP protocol recommendations to derive a daily intake schedule for at least one natural and/or at least one pharmaceutical agent.

The process will begin by collecting physiological and psychological data by traditional means including; medical questionnaires, bio/psycho/social questionnaires, medical history questionnaires. Psychological data can be collected by traditional Psychology testing including; the MMPI (Minnesota Multiphasic Personality Inventory), Luria-Nebraska Neuropsychological Battery, Hasltead-Reitan Neuropsychological Battery, WISC (Weschler Intellegence Scale for Children, WAIS (Weschler Adult Intelligence Scale, TOVA (Test of Variables of Attention), IVA Visual and Auditory Attention Testing, etc. A diagnosis of the subject's most severe condition is established by correlating the results to the DSM-IV (Diagnostic and Statistics Maual of Mental Disorders version 4) categories. Data will be collected from electro-magnetic information from all of the detectors on the Glasses (100), Wrist Device (200), and Finger Device (300). A diagnosis will be assessed using the subject's most severe condition, based on a DSM-IV category to the electro-magnetic physiological information. Both of the diagnoses will be correlated into a coherent diagnosis that will identify the most dominant condition. This condition will then be correlated to at least one natural and/or at least one pharmaceutical agent protocol. Its intended purpose is to increase the effectiveness of the device. The protocol is to be followed on a daily intake schedule.

1. An apparatus for pulsing light into at least one eye comprising:
a plurality of light sources arranged in an array having at least quadrants, each of the at least quadrants in the array having a plurality of light sources configured to independently emit light on different regions of the visual field of the at least one eye;
at least one physiology monitoring system for detecting at least one physiological state of a subject and producing at least one physiological output;
a control system to monitor the at least one physiological output from the at least one physiology monitoring system and for modulating the plurality of independent light sources in each of the at least quadrants in a predetermined light pulse combination or permutation in each of the at least quadrants based on the at least one physiological output, with the at least one physiological output correlated with different brainwave patterns and causing adjustment of the brainwave patterns through controlled light stimulation of various sections of the different regions of the visual field of the at least one eye.

2. The apparatus of claim 1 wherein the plurality of light sources in each of the at least quadrants are arranged in a plurality of different regions to allow production of light which is modulated independently within the different regions.

3. The apparatus of claim 1 wherein the at least one physiology monitoring system iteratively provides the at least one physiological output, and the control system generates a varied predetermined combination or permutation of light in each at least quadrant based on the successive iterative output of the at least one physiology monitoring system.

4. The apparatus of claim 1 wherein the at least one physiology monitoring system is selected from the group consisting of heart rate, heart pulse, muscle tension, skin temperature, skin conductance, Electroencephalogram or combinations thereof.

5. The apparatus of claim 1 wherein there are a plurality of physiology monitoring systems comprising at least Electroencephalogram, skin conductance and heart rate, and
wherein the control system generates the predetermined combination or permutation of light by combining at least one of the frequency, amplitude, coherence and phase of the input values from the physiology monitoring systems.

6. The apparatus of claim 1 wherein the physiology monitoring system includes a wrist detector.

7. The apparatus of claim 1 wherein the physiology monitoring system includes a head detector.

8. The apparatus of claim 1 wherein the physiology monitoring system includes a finger detector.

9. An apparatus for pulsing lights in the eye in conjunction with a natural or a pharmaceutical agent protocol regimen comprising:
a plurality of independent light sources arranged in an array having at least quadrants, each of the at least quadrants in the array having a plurality of light sources to emit light on different regions of the visual field of a subject;
at least one physiology monitoring system for detecting at least one physiological state of a subject and producing at least one physiological output;
a control system to monitor the at least one physiological output from the at least one physiology monitoring system and for modulating the plurality of independent light sources in the at least quadrants to independently emit light on different regions of the visual field of the at least one eye to stimulate various specific brain regions and generating information relating to natural or pharmaceutical agents for producing a physiological effect based on the at least one physiological output;
wherein the output from the at least one physiology monitoring system is used by the control system to determine the predetermined light pulse combination or permutation in each at least quadrant and a natural or pharmaceutical agent protocol regimen.

10. The apparatus of claim 9 wherein the plurality of light sources in each of the at least quadrants are arranged in a plurality of different regions to allow modulation of the light sources independently within the different regions.

11. The apparatus of claim 9 wherein the at least one physiological output from the at least one physiology monitoring system selected from the group consisting of heart rate, heart pulse, muscle tension, skin temperature, skin conductance, and an EEG or combinations thereof.

12. (canceled)

13. (canceled)

14. (canceled)

15. (canceled)

16. (canceled)

17. (canceled)

18. (canceled)

19. (canceled)

20. An apparatus for emitting light into at least one eye comprising:
a plurality of light sources supported in an array having at least quadrants, each of the at least quadrants in the array having a plurality of light sources,
a control system to individually modulate the plurality of independent light sources in the different at least quadrants, such that the light emitted from the plurality of light sources produces a predetermined combination or permutation of light in each of the at least quadrants to stimulate various specific brain regions.

21. The apparatus of claim 20 wherein the plurality of light sources in each of the at least quadrants are arranged in a plurality of different regions to allow modulation of the light sources independently within the different regions.

22. The apparatus of claim 20 wherein the at least quadrants include a central region including at least one light source and at least one peripheral region including at least one light source.

23. (canceled)

24. The apparatus of claim 20 wherein the plurality of light sources are caused to emit light with variable characteristics selected from the group consisting color, intensity, frequency and combinations thereof by the control system.

25. The apparatus of claim 20 wherein the plurality of light sources are caused to emit light in patterns having characteristics selected from frequency, phase, coherence, wave form, color of light, intensity of light or combinations thereof by the control system.

26. The apparatus of claim 1 wherein the physiology monitoring system iteratively produces the at least one physiological output, and the control system varies the predetermined light pulse combination or permutation in each of the at least quadrants as physiological activity changes.

27. The apparatus of claim 20, further comprising a wireless system to collect the information from the at least one physiology monitoring system and supplying such information to the control system and for wirelessly modulating the predetermined light pulse combination or permutation in each of the at least quadrants.
